(19) United States
(12) Patent Application Publication
(10) Pub. No.: US 2023/0200634 A1
(43) Pub. Date: Jun. 29, 2023

(54) PHOTODYNAMIC THERAPY IMAGING

(71) Applicant: MICRONVISION CORP., Bellevue, WA (US)

(72) Inventor: Xiaolong OUYANG, Bellevue, WA (US)

(21) Appl. No.: 18/113,395

(22) Filed: Feb. 23, 2023

Related U.S. Application Data
(63) Continuation-in-part of application No. 17/835,624, filed on Jun. 8, 2022.
Application No. 17/835,624 is a continuation-in-part of application No. 17/745,526, filed on May 16, 2022.
Application No. 17/745,526 is a continuation-in-part of application No. 17/473,587, filed on Sep. 13, 2021, now Pat. No. 11,330,973.
Application No. 17/473,587 is a continuation-in-part of application No. 17/362,043, filed on Jun. 29, 2021, now Pat. No. 11,350,816.
Application No. 17/473,587 is a continuation-in-part of application No. PCT/US2019/036060, filed on Jun. 7, 2019.
Application No. 17/473,587 is a continuation-in-part of application No. 16/363,209, filed on Mar. 25, 2019.
Application No. 17/473,587 is a continuation-in-part of application No. PCT/US2017/053171, filed on Sep. 25, 2017.
(60) Provisional application No. 63/346,377, filed on May 27, 2022.
(60) Provisional application No. 63/346,842, filed on May 28, 2022.
(60) Provisional application No. 63/347,659, filed on Jun. 1, 2022.
(60) Provisional application No. 63/218,362, filed on Jul. 4, 2021.
(60) Provisional application No. 63/213,499, filed on Jun. 22, 2021.
(60) Provisional application No. 63/210,034, filed on Jun. 13, 2021.
(60) Provisional application No. 63/218,362, filed on Jul. 4, 2021.
(60) Provisional application No. 63/213,499, filed on Jun. 22, 2021.
(60) Provisional application No. 63/210,034, filed on Jun. 13, 2021.
(60) Provisional application No. 63/197,639, filed on Jun. 7, 2021.
(60) Provisional application No. 63/197,611, filed on Jun. 7, 2021.
(60) Provisional application No. 63/183,151, filed on May 3, 2021.
(60) Provisional application No. 63/153,252, filed on Feb. 24, 2021.
(60) Provisional application No. 63/149,338, filed on Feb. 14, 2021.
(60) Provisional application No. 63/138,751, filed on Jan. 18, 2021.
(60) Provisional application No. 63/218,362, filed on Jul. 4, 2021.
(60) Provisional application No. 63/213,499, filed on Jun. 22, 2021.
(60) Provisional application No. 63/197,639, filed on Jun. 7, 2021.
(60) Provisional application No. 63/197,611, filed on Jun. 7, 2021.
(60) Provisional application No. 63/183,151, filed on May 3, 2021.
(60) Provisional application No. 63/153,252, filed on Feb. 24, 2021.
(60) Provisional application No. 63/149,338, filed on Feb. 14, 2021.
(60) Provisional application No. 63/138,751, filed on Jan. 18, 2021.
(60) Provisional application No. 63/129,703, filed on Dec. 23, 2020.
(60) Provisional application No. 63/124,803, filed on Dec. 13, 2020.
(60) Provisional application No. 63/121,924, filed on Dec. 6, 2020.
(60) Provisional application No. 63/121,246, filed on Dec. 4, 2020.
(60) Provisional application No. 63/107,344, filed on Oct. 29, 2020.
(60) Provisional application No. 63/087,935, filed on Oct. 6, 2020.
(60) Provisional application No. 63/083,932, filed on Sep. 27, 2020.
(60) Provisional application No. 63/077,675, filed on Sep. 13, 2020.
(60) Provisional application No. 63/077,635, filed on Sep. 13, 2020.
(60) Provisional application No. 63/183,151, filed on May 3, 2021.
(60) Provisional application No. 63/153,252, filed on Feb. 24, 2021.
(60) Provisional application No. 63/149,338, filed on Feb. 14, 2021.
(60) Provisional application No. 63/138,751, filed on Jan. 18, 2021.
(60) Provisional application No. 63/129,703, filed on Dec. 23, 2020.
(60) Provisional application No. 63/124,803, filed on Dec. 13, 2020.
(60) Provisional application No. 63/121,924, filed on Dec. 6, 2020.
(60) Provisional application No. 63/121,246, filed on Dec. 4, 2020.
(60) Provisional application No. 63/107,344, filed on Oct. 29, 2020.
(60) Provisional application No. 63/087,935, filed on Oct. 6, 2020.
(60) Provisional application No. 63/083,932, filed on Sep. 27, 2020.
(60) Provisional application No. 63/077,675, filed on Sep. 13, 2020.
(60) Provisional application No. 63/077,635, filed on Sep. 13, 2020.
(60) Provisional application No. 62/686,680, filed on Jun. 19, 2018.
(60) Provisional application No. 62/686,682, filed on Jun. 19, 2018.
(60) Provisional application No. 62/688,397, filed on Jun. 22, 2018.
(60) Provisional application No. 62/682,854, filed on Jun. 9, 2018.
(60) Provisional application No. 62/681,824, filed on Jun. 7, 2018.
(60) Provisional application No. 62/681,687, filed on Jun. 7, 2018.
(60) Provisional application No. 62/816,366, filed on Mar. 11, 2019.
(60) Provisional application No. 62/647,817, filed on Mar. 25, 2018.
(60) Provisional application No. 62/654,295, filed on Apr. 6, 2018.
(60) Provisional application No. 62/671,445, filed on May 15, 2018.
(60) Provisional application No. 62/816,366, filed on Mar. 11, 2019.
(60) Provisional application No. 62/399,429, filed on Sep. 25, 2016.
(60) Provisional application No. 62/399,436, filed on Sep. 25, 2016.
(60) Provisional application No. 62/399,712, filed on Sep. 26, 2016.
(60) Provisional application No. 62/405,915, filed on Oct. 8, 2016.
(60) Provisional application No. 62/423,213, filed on Nov. 17, 2016.
(60) Provisional application No. 62/424,381, filed on Nov. 18, 2016.
(60) Provisional application No. 62/428,018, filed on Nov. 30, 2016.
(60) Provisional application No. 62/429,368, filed on Dec. 2, 2016.
(60) Provisional application No. 62/485,454, filed on Apr. 14, 2017.
(60) Provisional application No. 62/485,641, filed on Apr. 14, 2017.
(60) Provisional application No. 62/502,670, filed on May 6, 2017.
(60) Provisional application No. 62/550,188, filed on Aug. 25, 2017.
(60) Provisional application No. 62/550,560, filed on Aug. 25, 2017.
(60) Provisional application No. 62/550,581, filed on Aug. 26, 2017.
(60) Provisional application No. 62/558,818, filed on Sep. 14, 2017.
(60) Provisional application No. 63/346,377, filed on May 27, 2022.
(60) Provisional application No. 63/346,842, filed on May 28, 2022.
(60) Provisional application No. 63/347,659, filed on Jun. 1, 2022.

(51) Int. Cl. A61B 1/04; A61B 1/303; A61B 1/00; A61B 1/06

(52) U.S. Cl. CPC A61B 1/043; A61B 1/303; A61B 1/00148; A61B 1/00032; A61B 1/00045; A61B 1/0638

(57) ABSTRACT

A photodynamic treatment device treating a patient's cervix opening with light that causes selected tissue to fluoresce and imaging the tissue while the treatment is in progress without affecting the treatment.

### REFERENCE TO RELATED APPLICATIONS

This application is a continuation-in-part of U.S. patent application Ser. No. 17/835,624 filed Jun. 8, 2022, which is a continuation in-part of U.S. patent application Ser. No. 17/745,526 filed May 16, 2022, which is a continuation-in-part of U.S. Pat. No. 17,473,587 filed Sep. 13, 2021 and now U.S. Pat. No. 11,330,973, which is a continuation-in-part of each of: U.S. patent application Ser. No. 17/362,043 filed Jun. 29, 2021 and now U.S. Pat. No. 11,350,816; International Patent Appl. No. PCT/US19/36060 filed Jun. 7, 2019; U.S. patent application Ser. No. 16/363,209 filed Mar. 25, 2019 and published as U.S. Pat. Appl. Publ. No. US2019/0216325; and International Patent Appl. No. PCT/US17/53171 filed Sep. 25, 2017.

This application claims the benefit of and incorporates by reference U.S. Provisional patent application Ser. No. 63/346,377 filed May 27, 2022, U.S. Ser. No. 63/346,842 filed May 28, 2022, and U.S. Ser. No. 63/347,659 filed Jun. 1, 2022.

This application incorporates by reference the entirety of the foregoing patent applications and claims the benefit of the filing date of each of the above-identified patent applications, as well as of the applications that they incorporate by reference, directly or indirectly, and the benefit of which they claim, including U.S. provisional applications, U.S. non-provisional applications, and international applications.

### FIELD

This patent specification generally relates to medical instruments for treating a patient's tissue with medication and light and to imaging in situ a treatment in progress.

### BACKGROUND

Photodynamic therapy (PDT) is a medical procedure that involves treating tissue with a photosensitizer and light, as discussed in U.S. Pat. Nos. 9,974,974 and 10,485,985 and in EPO publication WO 2010/078929, all of which are hereby incorporated by reference in this patent specification methods.

Said U.S. Pat. No. 10,485,985 discusses devices for photodynamic treatment of a patient's cervix. FIGS. 3 and 4 of this patent specification are a copy of FIGS. 3 and 4 of U.S. Pat. No. 10,485,985, which the patent states illustrate an example of such a device discussed in said patent publication WO 2010/078929. FIGS. 5 and 6 of this patent specification are a copy of FIGS. 5 and 6 of said U.S. Pat. No. 10,485,985, which are said therein to illustrate an example of an improved device of this type.

According to said U.S. Pat. No. 10,485,985, device 60 illustrated in FIGS. 3 and 4 is for use in photodynamic therapy of the cervix and comprises a housing 61a,61b with a resilient outer portion configured to press against the walls of the patient's vagina to hold the device in place such that a treatment surface 63 faces the cervix and a contact surface 63 covers the opening of the cervix. An LED lamp system 62 illuminates the treatment area at the cervix opening. Contact surface 63 serves as a drug delivery system. A battery 68 provides power and a control circuit 69 controls the illumination of the tissue being treated.

Further according to said U.S. Pat. No. 10,485,985, FIGS. 5 and 6 illustrate an improved device comprising a second housing portion 4 that is hollow and is configured to cover the cervix opening. A battery-powered LED system 22 irradiates tissue covered with housing portion 4, under the control of circuit on a board 24. The patent discusses FIGS. 3-6 thereof in more detail and identifies the other reference numerals in these figures.

To applicant's knowledge, an example of a device for photodynamic treatment of the cervix is offered commercially under the registered trademark Cevira. See https://photocure.com/cevira for an illustration of the commercial device and its placement over the cervix opening.

The photodynamic treatment devices discussed above typically are single-use and remains in place for an extended period, such as hours or days. During the treatment period, the light from the LED system can be at the same intensity or at different intensities at different times and may be continuous or intermittent, as controlled by a control circuit in the device.

### SUMMARY

The subject application describes an improvement that for the first time makes it possible to visually observe in situ the progress of treatment with photodynamic therapy and thus can enable health professionals and/or the patient to assess the progress of treatment and possibly adjust parameters such as the duration of treatment, timing and intensity of light impinging of the treatment area, etc.

To this end, one or more miniature cameras are incorporated in the device and configured to view the treatment area, or selection portions thereof, and to provide real time or intermittent image data to an outside display, through a cable or wirelessly.

As described in the initially presented claims but subject to amendments thereof in prosecuting this patent application, according to some embodiments a medical device for imaging fluorescence from tissue at or adjacent a patient's cervix being treated with photodynamic therapy comprises: a housing that has an open, cup-shaped distal portion configured for insertion to a treatment position in the patient's vagina to cover an external opening of the cervix and secure itself in place by contact with at least one of the patient's vagina and cervix for photodynamic therapy of patient's tissue; a power source, a light source, a camera system, and a control circuit integrated in said housing; wherein: said power source is configured to power said light source, camera system, and control circuit; said light source is configured to selectively illuminate tissue for photodynamic treatment and to cause fluorescence from portions thereof when the housing is at said treatment position relative to the cervix and the light source is energized; said camera system comprises a camera CamF1 configured to selectively image said fluorescence from selected tissue from a first viewpoint and provide first image data; and said control circuit is configured to selectively cause said power source to power said light source during one or more selected treatment time intervals and to cause said camera to image said selected tissue and to transmit said first image data at selected times; and a display operatively coupled with the camera CamF1 and configured to receive said first image data and display a first fluorescence image derived from said first image data.

According to some embodiments, the device can further comprise one or more of the following: (a) said camera system further comprises a camera CamF2 also integrated in said housing and configured to image said fluorescence from a second viewpoint and provide second image data, wherein said display is configured to receive said second image and to display a second fluorescence image derived from the second image data; (b) the device further includes a contact at said housing operatively coupled with said control circuit and a cable configured to releasably couple operatively with said contact for conveying said first and any second image data to said display; (c) the device further includes a user interface operatively coupled with said control circuit to control operation of one or more of said cameras CamF1 and CamF2 and said light source by operator commands; (d) the device further includes a wireless link between said control circuit and said display configured to selectively transmit wirelessly said first and any second image data to said display; (e) said control circuit is configured to energize said light source only intermittently, at selected times while the housing is in said treatment position; (f) said control circuit is configured to energize said light source at respective different intensities at selected times while the housing is in said treatment position; (g) said control circuit is configured to energize said light source to illuminate said tissue with light at respective different wavelength ranges at selected times while the housing is in said treatment position; and (h) said selected time intervals can include one or more time intervals of illumination with white light or one or more time intervals of illumination with light in other wavelength ranges narrower that the white light range.

According to some embodiments, a medical device for imaging fluorescence from a patient's tissue treated with photodynamic therapy comprises: a housing configured for insertion to a treatment position in the patient to cover selected internal tissue of the patient and remain in place during a photodynamic therapy period exceeding an hour; a power source, a light source, a camera system, and a control circuit integrated in said housing; wherein: said power source is configured to power said light source, camera system, and control circuit; said light source is configured to selectively illuminate said selected internal tissue for photodynamic treatment and to cause fluorescence from portions thereof when the housing is at said treatment position and the light source is energized; said camera system comprises a camera CamF1 configured to selectively image said fluorescence from a first viewpoint and provide first image data; and said control circuit is configured to selectively cause said power source to power said light source during one or more time intervals during said treatment period and to cause said camera system to transmit said first image data; and a display operatively coupled with the camera CamF1 and configured to receive said first image data and display a first fluorescence image derived from said first image data.

The medical device described in the immediately preceding paragraph can further comprise one or more of the following: (a) the selected internal tissue is at the entrance of the patient's cervix and the housing has an open, cup-shaped portion configured to conform to and cover tissue at the cervix entrance; and (b) said camera system further comprises a camera CamF2 also integrated in said housing and configured to image said fluorescence from a second viewpoint and provide second image data, wherein said display is configured to receive said second image and to display a second fluorescence image derived from the second image data.

According to some embodiments, a method of photodynamic therapy of internal tissue of a patient medicated to fluoresce when illuminated with selected light comprises: inserting a housing of a treatment device at a treatment position adjacent said internal tissue of the patient and maintaining the housing in the treatment position for treatment period of an hour or more; illuminating said internal tissue with light from a light source in said housing while said housing is in said treatment position to cause at least selected portions of the tissue to fluoresce; imaging said fluorescence from a first viewpoint with a camera CamF1 integrated in said housing of the treatment device while the housing is in said treatment position in the patient to provide first image data; transmitting said first image data to a display outside the patient and displaying the first image data at said display; and controlling at least one of said illuminating and imaging with at least one of a control circuit integrated in said housing and a user interface device that is outside the patient.

The method can further comprise one or more of the following: (a) imaging said fluorescence from a second viewpoint with a camera CamF2 integrated in said housing of the treatment device while the housing is in said treatment position in the patient to provide second image data and transmitting the second image data to said display and displaying the second image data at said display; and (b) at least one of said transmitting and said controlling is via a wireless link between the control circuit and an external interface.

### BRIEF DESCRIPTION OF THE DRAWINGS

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims. The subject matter hereof will be described and explained with additional specificity and detail through use of the accompanying drawings in which:

FIG. 1 is a perspective view of a photodynamic treatment device incorporating one or more cameras and associated electronics, according to some embodiments.

FIG. 2 is a perspective view of a portion of the photodynamic treatment device from another viewpoint and of external display and user interface facilities, according to some embodiments.

FIGS. 3 and 4 illustrate a prior art device for photodynamic treatment of a cervix.

FIGS. 5 and 6 illustrate another prior art device for photodynamic treatment of a cervix.

FIG. 7 illustrates a cross-section (not to scale) of an improved device for photodynamic treatment and concurrent in situ imaging of a patient's cervix, according to some embodiments.

FIG. 8 is a schematic representation of an improved device for photodynamic treatment and concurrent imaging of a patient's cervix showing associated cameras, control circuit and outside display and user interface facilities, according to some embodiments.

### DETAILED DESCRIPTION

A detailed description of examples of preferred embodiments is provided below. While several embodiments are described, the new subject matter described in this patent specification is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all these details. Moreover, for the purpose of clarity, certain technical material that is known in the related art has not been described in detail to avoid unnecessarily obscuring the new subject matter described herein. It should be clear that individual features of one or several of the specific embodiments described herein can be used in combination with features of other described embodiments or with other features. Like reference numbers and designations in the various drawings indicate like elements.

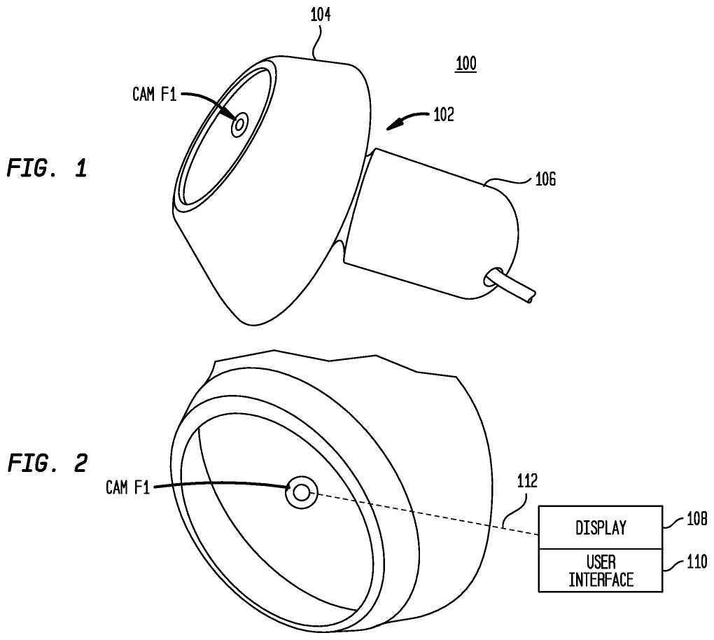

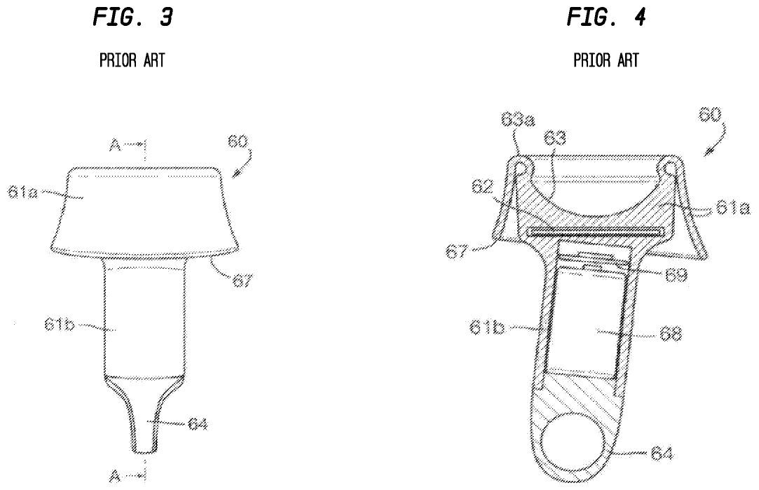

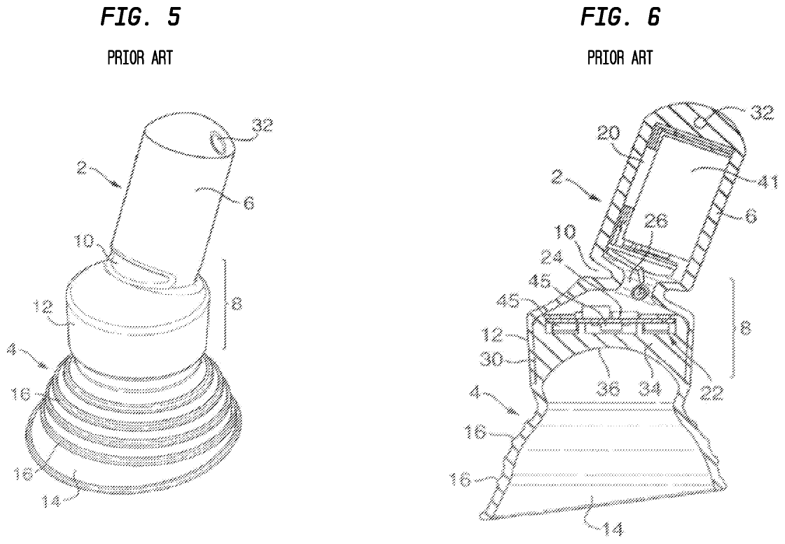

FIGS. 1 and 2 illustrate in perspective an improved integrated device 100 for photodynamic treatment and concurrent imaging of a patient's cervix. Device 100 can be placed at a treatment position relative to the patient's cervix as discussed in the Background section above for the prior art treatment device of U.S. Pat. Nos. 9,974,974 and 10,485,985 and of EPO publication WO 2010/078929. Device 100 comprises a housing 102 that has a distal portion 104 and a proximal portion 106 that can be like the housing comprising distal portion 4 and proximal portion 2, respectively, in the prior art device illustrated in FIGS. 5 and 6. Device 100 further comprises a camera CamF1 that the prior art device lacks, and may comprise an additional camera CamF2, not seen in FIGS. 1 and 2, as well as other elements not present in the prior art and described below. Device 100 communicates with an outside display 108 and an outside user interface 110 via a link 112 that can be a cable or a wireless link.

FIG. 7 illustrates device 100 in cross-section but not to scale, when in a treatment position relative to a schematically illustrated opening of a cervix 101 and vagina walls 103. As illustrated in FIG. 7, housing 102 of device 100 comprises a distal portion 104 and a proximal portion 106. Proximal portion 106 preferably contains a power source 702 such as battery. Distal portion 104 comprises an open, cup-shaped portion 704 that, when in a treatment position, faces and is adjacent to cervix 101. The inner side of housing portion 704 incorporates a light source 706 that typically comprises one or more LEDs. A circuit board 708 contains a control circuit 710 (described further below) and is operatively coupled with power source 702 and light source 706 through suitable switching circuits (not shown) and with a camera system comprising cameras CamF1 and CamF2 that are integrally mounted to portion 704 to view cervix 101 from respective viewpoints. Light source 706 is configured to selectively illuminate tissue at cervix 101 for photodynamic treatment and to cause fluorescence from portions thereof when device 100 is at a treatment position relative to cervix 101 and light source 706 is energized, as in said prior art device. Cameras CamF1 and CamF2, which said prior art devices do not have, are configured to selectively image said fluorescence from selected tissue at cervix 101 from a first viewpoint and a second viewport and provide first image data and second image data, respectively. Control circuit 710 is configured to selectively cause power source 702 to power and thus energize light source 706 during one or more treatment time intervals and to cause cameras CamF1 and CamF2 to image tissue at cervix 101 and to transmit first and second image data to display 108 at selected times. Display 108 is operatively coupled with the camera CamF1 and camera CamF2 and is configured to receive said first image data and said second image date and to display a first fluorescence image derived from said first image data and a second fluorescence image derived from the second image data.

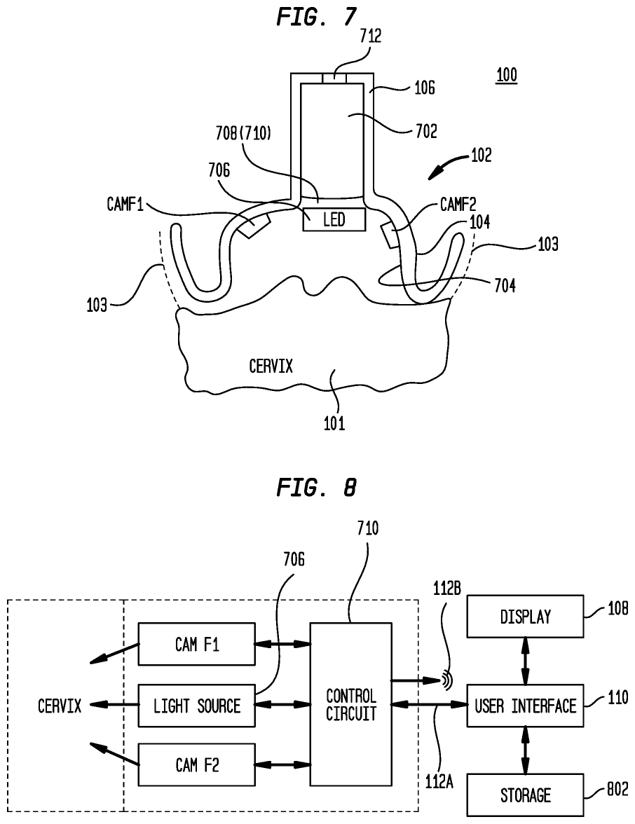

Medical device 100 can be configured to include only camera CamF1 or only camera CamF2, or to include more than two cameras viewing tissue at cervix 101 from respective viewpoints to thereby provide a stereo view or additional views of the tissue. Display 108 and/or user interface 110 can be equipped with a storage facility 802 (FIG. 8) to selectively store image data from cameras CamF1 and from additional cameras that device 100 may contain. If a cable link 112a (FIG. 8) is used to transmit image date out of device 100, proximal portion 102 can be provided with an electrical contact 712 operatively coupled to circuit board 706. Cable 112a is configured to releasably attach to contact 712 when transmission of images from device 100 is desired.

FIG. 8 is a schematic representation of internal components of device 100 for photodynamic treatment and concurrent in situ imaging of a patient's cervix according to some embodiments. As illustrated, light source 706 delivers light to tissue 101 and cameras CamF1 and CamF2 view the tissue from respective viewpoints when device 100 is in its treatment position relative to cervix 101. Control circuit 710 controls the operation of the light source and the cameras, the transmission of image data from the cameras to display 108, and interaction with user interface 110. If desired, display 108 and/or user interface 110 can contain a storage facility 802 for storing image data and/or other data associated with operation of device 100. Components 108, 110 and 802 can be separate units or two of them or all three can be integrated into a single unit.

In use, after device 100 is placed in the treatment position relative to cervix 101 as described above, the photodynamic treatment can proceed in different ways. For example, device 100 can be powered before insertion in the treatment position such that light source 707 continuously emits light for a treatment that typically lasts more than an hour and can extend over days. As another example, control circuit 710 can be configured to energize light source 706 intermittently for time periods that have selected durations and are separated by time periods of selected durations during which light source 706 is turned OFF. As another example, light source 710 can be turned ON and/or OFF by operator commands entered at user interface 110 and executed through control circuit 710, or different modes of operation of light source 706 can be programmed into control circuit 710 and executed automatically or when prompted by commands entered through user interface 110.

Light source 706 can be configured to emit light only in wavelength ranges selected to cause tissue to fluoresce. Such wavelength ranges can be selected in consideration of agents or medication delivered to the tissue being treated. In another example, light source 706 can be configured to emit treatment light at different wavelength ranges at different times, or different wavelength ranges concurrently, as commanded by control circuit 710. As another example, light source 706 can be configured to emit white light at selected times concurrently with said treatment light or when the treatment light is turned OFF, under control from control circuit 710. As another example, light source 706 can be configured to emit light at different intensities at different times, under control from control circuit 710.

Cameras CamF1 and any additional cameras can be providing image data continuously. However, control circuit 710 preferably is configured to command the cameras to provide image data only at selected times or selected time intervals. For example, control circuit 710 can be configured to command camera CamF1 and any additional cameras to provide a still image or a video clip every few minutes or every hour at some other time points during photodynamic treatment of a patient. In one example, control circuit 710 issues such commands to the camera(s) automatically, based on internal programming. In another example, control circuit 710 issues such commands when commanded by a user through user interface 110 and link 112. Preferably, control circuit 710 is configured with the facility to be re-programmed by commands from user interface 110.

Image data from the camera(s) can be stored in storage facility 802 in several different modes. One mode is to automatically store all image data sent out from device 100. Another is to store image data provided only at selected times during a photodynamic treatment of a patient, for example once every few minutes or once per hour or day, at times that are regularly or irregularly spaced from each other. Yet another is to store image data only when commanded by instructions stored in control circuit 710 or sent thereto from user interface 110. Storage facility 802 may be configured to be worn by a patient during photodynamic treatment, in which case facility 802 may be independently powered and preferably wirelessly connected to device 100.

Cameras CamF1 and CamF2 and any additional cameras can be configured to image only or primarily fluorescence from the tissue subjected to photodynamic treatment. In another example, while one or more of the cameras can image fluorescence, one or more other cameras in device 100 can be configured to image white light and/or light at wavelength ranges different from those used for photodynamic treatment. In yet another example, one or more of the cameras in device 100 can be configured to image primarily fluorescence some of the time and image white light and/or light in other narrower ranges at other times, for example by changing light filter parameters of the cameras electronically by commands from control circuit 710 or user interface 110. White light images, or images at certain wavelength ranges that are narrower and differ from the treatment wavelength ranges, can be particularly useful in photodynamic treatment because white light images and/or images in narrower wavelength ranges different from fluorescence ranges can be significantly clearer that fluorescence images and can provide health professionals with important information in addition to that fluorescence images provide.

Examples of cameras and light sources that can be used for CamF1 and other cameras and for light source 706 are described in more detail in said U.S. Pat. No. 11,330,973 and said application Ser. No. 17/835,624, which are incorporated by reference.

Device 100 has been described above as applied to photodynamic treatment and imaging of cervical tissue, but it should be clear that it can be applied to such treatment of other organs.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

What it claimed is:

1. A medical device for imaging fluorescence from tissue at or adjacent a patient's cervix treated with photodynamic therapy, comprising:
a housing that has an open, cup-shaped distal portion configured for insertion to a treatment position in the patient's vagina to cover an external opening of the cervix and secure itself in place by contact with at least one of the patient's vagina and cervix for photodynamic therapy of patient's tissue;
a power source, a light source, a camera system, and a control circuit integrated in said housing;
wherein:
said power source is configured to power said light source, camera system, and control circuit;
said light source is configured to selectively illuminate tissue for photodynamic treatment and to cause fluorescence from portions thereof when the housing is at said treatment position relative to the cervix and the light source is energized;
said camera system comprises a camera CamF1 configured to selectively image said fluorescence from selected tissue from a first viewpoint and provide first image data; and
said control circuit is configured to selectively cause said power source to power said light source during one or more treatment time intervals and to cause said camera system to image said selected tissue and to transmit said first image data at selected times; and
a display operatively coupled with the camera CamF1 and configured to receive said first image data and display a first fluorescence image derived from said first image data.

2. The medical device for imaging fluorescence from a patient's cervix of claim 1,
wherein said camera system further comprises a camera CamF2 also integrated in said housing and configured to image said fluorescence from a second viewpoint and provide second image data, wherein said display is configured to receive said second image and to display a second fluorescence image derived from the second image data.

3. The medical device of claim 2, further including a contact at said housing operatively coupled with said control circuit and a cable configured to releasably couple operatively with said contact for conveying said first and second image data to said display.

4. The medical device of claim 3, further including an outside user interface operatively coupled with said control circuit to control operation of one or more of said cameras CamF1 and CamF2 and said light source by operator commands.

5. The medical device of claim 2, further including a wireless link between said control circuit and said display configured to selectively transmit wirelessly said first and second image data to said display.

6. The medical device of claim 5, further including an outside user interface operatively coupled through said wireless link with said control circuit to control operation of one or more of said cameras and light source by operator commands.

7. The medical device of claim 1, further including a contact at said housing operatively coupled with said control circuit and a cable configured to releasably couple operatively with said contact for conveying said first image data to said display.

8. The medical device of claim 7, further including an outside interface operatively coupled with said control circuit to control operation of one or more of said camera CamF1 and said light source by operator commands.

9. The medical device of claim 1, further including a wireless link between said control circuit and said display configured to selectively transmit wirelessly said first image data to said display.

10. The medical device of claim 9, further including an outside user interface operatively coupled through said wireless link with said control circuit to control operation of one or more of said camera CamF1 and light source by operator commands.

11. The medical device of claim 1, in which said control circuit is configured to energize said light source only intermittently, at selected times while the housing is in said treatment position.

12. The medical device of claim 1, in which said control circuit is configured to energize said light source at respective different intensities at selected times while the housing is in said treatment position.

13. The medical device of claim 1, in which said control circuit is configured to energize said light source to illuminate said tissue with light at respective different wavelength ranges at different selected times while the housing is in said treatment position.

14. The medical device of claim 1, in which said control circuit is configured to energize said light source to illuminate said tissue with light at respective different wavelength ranges at selected time intervals while the housing is in said treatment position, wherein said selected time intervals include time intervals of illumination with white light or time intervals of illumination with light in wavelength ranges narrower that the white light range.

15. A medical device for imaging fluorescence from a patient's tissue treated with photodynamic therapy, comprising:
a housing configured for insertion to a treatment position in the patient to cover selected internal tissue of the patient and remain in place during a photodynamic therapy period exceeding an hour;
a power source, a light source, a camera system, and a control circuit integrated in said housing;
wherein:
said power source is configured to power said light source, camera system, and control circuit;
said light source is configured to selectively illuminate said selected internal tissue for photodynamic treatment and to cause fluorescence from portions thereof when the housing is at said treatment position and the light source is energized;
said camera system comprises a camera CamF1 configured to selectively image said fluorescence from said tissue from a first viewpoint and provide first image data; and
said control circuit is configured to selectively cause said power source to power said light source at selected time intervals during said treatment period and to cause said camera system to image said tissue and to transmit said first image data; and
a display operatively coupled with the camera CamF1 and configured to receive said first image data and display a first fluorescence image derived from said first image data.

16. The medical device of claim 15, in which the selected internal tissue is at the entrance of the patient's cervix and the housing has an open, cup-shaped portion configured to conform to and cover tissue at the cervix entrance.

17. The medical device of claim 16, wherein said camera system further comprises a camera CamF2 also integrated in said housing and configured to image said fluorescence from a second viewpoint and provide second image data, wherein said display is configured to receive said second image and to display a second fluorescence image derived from the second image data.

18. A method of photodynamic therapy of internal tissue of a patient medicated to fluoresce when illuminated with selected light, comprising:
inserting a housing of a treatment device at a treatment position adjacent said internal tissue of the patient and maintaining the housing in the treatment position for treatment period of an hour or more;
Illuminating said internal tissue with light from a light source in said housing while said housing is in said treatment position to cause at least selected portions of the tissue to fluoresce,
imaging said fluorescence from a first viewpoint with a camera CamF1 integrated in said housing of the treatment device while the housing is in said treatment position in the patient to provide first image data;
transmitting said first image data to a display outside the patient and displaying the first image data at said display; and
controlling at least one of said illuminating and imaging with at least one of a control circuit integrated in said housing and an interface device that is outside the patient.

19. The method of claim 18, further comprising imaging said fluorescence from a second viewpoint with a camera CamF2 integrated in said housing of the treatment device while the housing is in said treatment position in the patient to provide second image data and transmitting the said second image data to said display and displaying the second image data at said display.

20. The method of claim 19, in which at least one of said transmitting and said controlling is via a wireless link between the control circuit and an external interface.
